(19) United States
(12) Patent Application Publication
(10) Pub. No.: US 2007/0258937 A1
(43) Pub. Date: Nov. 8, 2007

(54) Deodorizing device

(76) Inventors: Steven A. Bolkan, Hopewell, NJ (US); Raymond F. Ashley, Pennington, NJ (US)

Correspondence Address: CHURCH & DWIGHT CO., INC., LAW DEPT. - PATENTS, 469 NORTH HARRISON STREET, PRINCETON, NJ, 08543-5297, US

(21) Appl. No.: 11/418,334

(22) Filed: May 4, 2006

(51) Int. Cl. A61L 9/00

(52) U.S. Cl. 424/76.2

(57) ABSTRACT

The present invention is directed to a deodorizing device and a method for forming the deodorizing device. The deodorizing device comprises a deodorizing composition and an adhesive, such as a hotmelt adhesive, adhered between two nonwoven gas porous sheets. The deodorizing composition includes deodorizing particles such as sodium bicarbonate and zeolites. The method of making the deodorizing device of the present invention includes loading the deodorizing composition and adhesive on a first nonwoven gas porous sheet, such as a nonwoven gas porous polyester material, heating the first nonwoven gas porous sheet, and adhering a second nonwoven gas porous sheet to the first nonwoven gas porous sheet possessing the deodorizing composition and adhesive. The deodorizing device can be manipulated for use in a variety of applications.

### FIELD OF THE INVENTION

The present invention relates to deodorizing material. Specifically, the present invention relates to a novel deodorizing article and method of making the same.

### BACKGROUND OF THE INVENTION

The air contains undesirable visible and invisible foreign materials some of which can impart a variety of odors. These odor emitting materials come from a wide range of sources such as cigars and cigarettes, animal and human fluids and gases, mildew, cooking, closets, gym bags, wardrobes, dresser drawers, dust masks, pillows, bedding, mattresses, clothing, industrial by products, organic gases generated by chemical materials including adhesives and paints, gases generated by a variety of manufactured products such as home appliances (e.g. vacuums, refrigerators), cars, planes, trains, etc.

The suppression or elimination of odors, particularly undesirable odors, has been the objective of untold investigations. In general, these investigations have been focused on either of two approaches, namely (a) odor masking, in which a substance of strong yet relatively pleasant odor is introduced into the proximity of a less pleasant odor source with the intent of overburdening the olfactory receptors with the dominant pleasant odor, or (b) sequestering the undesired odorous substance in a non-volatile form either by chemical reaction, adsorption or absorption on a sorbent material exhibiting a sorptive preference for the odorous substance.

Food and beverage spills are common odor producing materials in and around a kitchen that often require odor control via either odor masking or odor sequestering. This is especially true in refrigerators and waste receptacles in kitchens. Many times, a spill or a leak from a food or beverage container in a refrigerator goes unnoticed until the user of the refrigerator desires the item which has been spilled or which has leaked. By the time the item is desired, the spill or leak usually has solidified or has begun to solidify, leaving a hard to remove stain or residue on the refrigerator shelf or drawer. Often, this stain or residue will emit malodorous aromas into the refrigerator. The same is true for food stored in cabinets. Thus, a need is present in the art to provide an effective means for providing odor control in refrigerators and cabinets.

Trash receptacles also present problems with odor control. In the case of trash receptacles, a food or beverage containing bottle, can, jar, or resealable package, containing residual food or beverage is often thrown into a trash receptacle. This residual food or beverage content will often leak into the trash container and emit odors. Most trash receptacles are lined with plastic garbage bags; however the bags do not provide an effective means for controlling odors from discarded food items. Also, when the plastic bags are compromised, e.g. damaged during installation, use or removal, any foods in the plastic bag may escape into the trash receptacle and will often solidify or began to solidify leaving a residue, which will emit malodorous aromas. As will be recognized, the same issues are faced with disposal of nonconsumable trash, such as, for example, pet litter. Therefore, there is also a need in the art to provide an effective odor control means in trash receptacles.

In addition, various foods stored in a refrigerator or cabinets often emit strong aromas. For example, aromas from garlic, cheeses, meats and/or spices will often permeate a refrigerator when these food items or dishes containing these food items are stored in a refrigerator, even for a short period of time. Many times, the aroma from one food item will blend with the aroma or flavor of another food item or overpower the aroma or flavor of another food item stored in the refrigerator. Likewise, spices and other food items, such as coffee, will often permeate a cabinet in which these items are stored. This often results in the wasting of food, due to the loss of flavor or appeal of a food item which has been overpowered by the aroma or flavor of another food item.

Many techniques have been employed to reduce food odor problems over the years. The most frequent technique is to carefully wrap food items, for example with plastic films, prior to storage in the refrigerator or cabinet. However, some odors or aromas from the wrapped food may still escape, due to various reasons, such as, incomplete wrapping, odors too strong to be effectively contained, or damage to the wrapping due to movement of food items in and out of the refrigerator and/or cabinet.

Other techniques used to reduce this problem include placing an open box of baking soda or other odor controlling substance in the refrigerator or cabinet. For instance, U.S. Pat. No. 4,624,366, issued to the present assignee, discloses a container containing powdered material capable of absorbing odors in a confined area. The '366 container has at least one side portion that is covered with gas permeable membrane to expose materials in the container to exterior gases such as the atmosphere while keeping the material in the container. The object of the '366 container is to provide a stable container that will retain its contents when tipped.

Another example of prior art odor adsorbing devices includes U.S. Pat. No. 5,046,604 to Forhetz et al. directed to an odor adsorbing liner that has pouches of odor adsorbing particles between two sheets. The pouches are prepared by stitching the sheets together in a quilt-type fashion. The sheets of material are disclosed as paper or cloth. The product of the '604 patent suffers from the problem that the odor absorbing particles are loose in the pouches and if one of the sheets becomes tom, the particles can be released into the refrigerator or cabinet. Further, the liners are difficult to cut to a specific size for a particular shelf or drawer since the areas containing the loose particles cannot be cut. If these sheets were cut in the pouch area, the loose particles in the pouch would no longer be contained within the pouch area, thereby causing the particles to be removed from the liner, which in turn will reduce the effectiveness of the odor adsorbing liner of the '604 patent.

Other prior art methods include a device having nestable front and back panels which nest to hold a removable pouch of odor adsorbing particles. Such devices are described in U.S. Pat. Nos. 5,772,959 and 5,468,447 both to Bermas. The odor adsorbing particles of these patents are sealed within a porous paper or a nonwoven polymeric felt. The device of these patents is effective in controlling odor within confined spaces.

Attempts have been made in the art to control odors in containers, such as soiled diaper storage containers. U.S. Pat. No. 5,022,553 to Pontius discloses a single nonwoven liner for a diaper container, wherein the single nonwoven liner is impregnated with an odor adsorbing material.

Deodorizing devices are still desired to provide effective and easy to use odor control in a variety of applications. Efficient methods of making such devices are also needed to reduce the cost of such devices.

### SUMMARY OF THE INVENTION

The present invention provides a deodorizing device containing a deodorizing composition and a method of producing the deodorizing device containing the deodorizing composition. The deodorizing device comprises a pair of nonwoven gas porous sheets, an adhesive to adhere the sheets together and the deodorizing composition placed between the nonwoven sheets. The method of preparing the deodorizing device includes introducing the deodorizing composition onto a first nonwoven gas porous sheet material, adding or incorporating adhesive to the deodorizing composition, covering the deodorizing composition and the adhesive by a second nonwoven gas porous sheet material, and activating the adhesive to secure the first and second porous sheets together.

### BRIEF DESCRIPTION OF THE DRAWING

FIG. 1: is a diagram of the process for making the deodorizing device of the present invention.

### DETAILED DESCRIPTION OF THE INVENTION

The present invention is a deodorizing device and a method for making the deodorizing device. The device includes a nonwoven gas porous sheet product containing a deodorizing composition. The deodorizing composition can be largely a single component or a mixture of components. The deodorizing composition is disposed on a first nonwoven gas porous sheet material such as a nonwoven polyester fabric and then covered with a second nonwoven gas porous sheet material to produce the deodorizing device. The respective sheet materials are adhered together by an adhesive contained with the deodorizing composition. Once formed, the deodorizing device can be manipulated to be used in a wide variety of applications. The deodorizing device of this invention has particular use for placement in confined areas such as in storage spaces and the like to deodorize the enclosed space.

Suitable deodorizing compositions include components or deodorizing particles being any material known to those skilled in the art which will effectively control, neutralize and/or absorb odor. These deodorizing particles can be used alone or in combination with one another and form the deodorizing composition. Examples of such materials include, but are not limited to sodium bicarbonate, zeolites, activated carbons, activated charcoal, diatomaceous earths, cyclodextrin, quaternary ammonium salts, silane quaternary ammonium salts, clays, fragrance oils and the like. Fragrance oils may be added to the composition as liquids or while being supported on inert carriers.

Suitable amounts for each of the deodorizing particles forming the deodorizing composition include about 50-100 wt. % of sodium bicarbonate, about 0 to 10 wt. % of zeolites, about 0 to 20 wt. % of activated carbon, about 0 to 5 wt. % of quaternary ammonium salts, about 0 to 5 wt. % of the silane quartemary ammonium salts and about 0 to 2 wt. % of fragrance oils. Regardless of how much of the individual components are used, the total amounts of the deodorizing particles used to form the deodorizing composition will equal 100%. The final composition may take any form including powders or pellets.

The sodium bicarbonate employed herein is readily available commercially. Arm & Hammer°0 baking soda, including grades 2 or 5 are particularly useful. The zeolite employed herein and useful in odor removal is a molecular sieve adsorbent that selectively adsorbs molecules on the basis of the size and shape of the adsorbate molecule. Zeolites adsorbents are well known. Smellrite® molecular sieve material obtained from UOP LLC, Des Plaines, Ill. is exemplified. Smellrite® is a synthetic sodium aluminum silicate with a zeolite structure that has been treated to be absorptive of odoriferous compounds. This particular molecular sieve works by having organophilic micropores which attract odor molecules and trap them within its porous structure to effectively remove them from the environment.

To the deodorizing particles listed above, an adhesive is mixed with or placed onto the deodorizing composition to secure the sheets together and maintain the deodorizing composition within the deodorizing device. The adhesive can be pressure sensitive, hotmelt or solvent activated. Preferably, a hotmelt adhesive in the form of non-sticky particules is included in the deodorant composition. An example of a hotmelt adhesive is Griltex® hotmelt, a product of EMS Griltech, Sumter, S.C. Griltex® is a heat-resistant, polymer-containing, solvent free binder. The adhesive is based on copolyamide and copolyester chemistry and can be in the form of powder or pellets. The adhesive can be present in about 2-6 wt. % of the deodorant composition. In some cases the adhesive added to the composition will be increased to about 5-15 wt. %, preferably about 5-10 wt. % to better adhere the deodorizing composition to the deodorizing device. If used herein, the hotmelt adhesive is heated to a temperature wherein the adhesive melts to fuse the deodorizing particles into the deodorizing device and allows the opposing sheets to adhere to each other and maintain the deodorizing particles there-between. The device serves as a carrier for the deodorizing composition, so that such device may be left in spaces to be deodorized without leaving behind dust, residue or unnecessary debris.

The nonwoven gas porous material may be a nonwoven fabric or nonwoven web. As used herein the term “nonwoven fabric” or “nonwoven web” means a web having a structure of individual fibers or threads which are interlaid, but not in an identifiable manner as in a knitted or meshed fabric. The nonwoven structures are in sheet form and are gas porous. Nonwoven fabrics or webs have been formed from many processes such as for example, meltblowing processes, spunbonding processes, air-laid and bonded-carded web processes.

In the meltblowing process, fibers are generally formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. As used herein, the term “microfibers” means small diameter fibers. The meltblown fibers are generally carried by the high velocity gas stream and are deposited on a collecting surface to form a web of randomly dispersed meltblown fibers. Such a process is disclosed, for example, in U.S. Pat. No. 3,849,241 to Butin et al. and U.S. Pat. No. 5,213,881 to Timmons et al. Meltblown fibers are often microfibers which can be continuous or discontinuous and can be tacky when deposited onto a collecting surface.

In the spunbonding process, fibers are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman; U.S. Pat. No. 3,542,615 to Dobo et al.; and U.S. Pat. No. 5,382,400 to Pike et al.; the entire content of each is incorporated herein by reference. Spunbond fibers are generally not tacky when they are deposited onto a collecting surface and thus often require additional mechanical or chemical bonding to form an integrated stabilized web.

Thermoplastic fibers are used to produce the nonwoven material of the present invention and can be prepared from any thermoplastic polymer material. Non-limiting examples of polymeric materials which can be utilized for forming fibers include polyolefin polymers such as polypropylene, polybutylene and polyethylene, nylon, rayon, cellulosic and blends thereof, including blends with cotton.

The most preferred polymer materials used to form the thermoplastic fibers include polyesters. More preferred polyesters are based on polyethylene terephthalate homopolymers, polybutylene terephthalate homopolymers, polyethylene terephthalate/polybutylene terephthalate copolymers, polyethylene terephthalate copolymers, polyethylene terephthalate/polybutylene terephthalate mixtures and/or mixtures thereof, although any other polyesters can be used as well, either alone or in any combination with any polyester. Polyethylene terephthalate (PET) and polybutylene terephthalate (PBT) are often just called polyester. Polyethylene terephthalate is widely used as an extrusion and injection-molding resin and is used to make a variety of household or industrial articles, including appliance parts, containers, and auto parts.

Polybutylene terephthalate is chemically similar (in both composition and properties) to polyethylene terephthalate, but polybutylene terephthalate has a lower melting point and thus can be processed at lower temperatures. Polybutylene terephthalate is a semi-crystalline, white or off-white polyester that crystallizes more rapidly than polyethylene terephthalate. It has somewhat lower strength and stiffness than polyethylene terephthalate, but is a little softer and has higher impact strength.

The individual nonwoven gas porous sheet materials may have different qualities and purposes when forming the completed device. The first nonwoven sheet material may serve as a bottom layer of the deodorizing device. The second nonwoven sheet material used herein can be hydrophilic, and may serve as a top layer of the deodorizing device. The second nonwoven sheet material can be a “scrim” to minimize dustiness of the product. In comparison to the top layer, the bottom layer is a heavier sponge-like nonwoven material. The device of the present invention uses the heavier sponge-like bottom layer to receive the deodorizing composition, while the lighter top layer adheres to the bottom layer and serves to allow good air flow.

Each of the individual nonwoven gas porous sheet materials have a thickness of about 0.05 to 6 mm and an optimal weight of about 25 to 200 grams per square meter. The first nonwoven sheet material typically will have a thickness of about 0.5-4 mm, preferably about 1-3 mm. The second nonwoven sheet material typically will have a thickness of about 0.05 to 2 mm, preferably about 0.1 to 1.0 mm. The completed deodorizing device product may have a thickness range of about 0.50 mm-5 mm, preferably 1 mm-3 mm.

In manufacturing the deodorizing composition the component(s) are pre-weighed to form the deodorizing composition. For example, a deodorizing composition used in the present invention can include a mixture of sodium bicarbonate, a zeolite and a hotmelt adhesive, all of which are in powder form. Once weighed, the components are placed in a high shear powder mixer such as a ribbon blender to ensure proper mixing. Prior to blending the components, care is taken to ensure the mixer is clean and free of all foreign matter and fragrance. The components may be placed in the mixer in any order however, a suitable order for placing the components in the mixer is: 1) sodium bicarbonate, 2) zeolite and 3) adhesive. The components may be mixed for any amount of time appropriate for blending the components together, e.g. ten minutes of mixing.

The deodorizing device comprises the deodorizing composition and the adhesive adhered between two nonwoven gas porous sheets. Specifically, the deodorizing composition and the adhesive is applied onto a first nonwoven gas porous sheet material, which forms a bottom layer and is then covered with a second nonwoven gas porous sheet material, which forms a top layer. Prior to covering with the second sheet, the adhesive is activated, e.g. heated, and serves to join the two nonwoven gas porous sheets together as well as adhere the deodorizing composition to the nonwoven gas porous sheets. The nonwoven sheet materials serve as a carrier for the deodorizing composition. Due to its specific construction, the deodorizing device can reside in any sized space requiring deodorizing without leaving behind dust, residue or unnecessary debris.

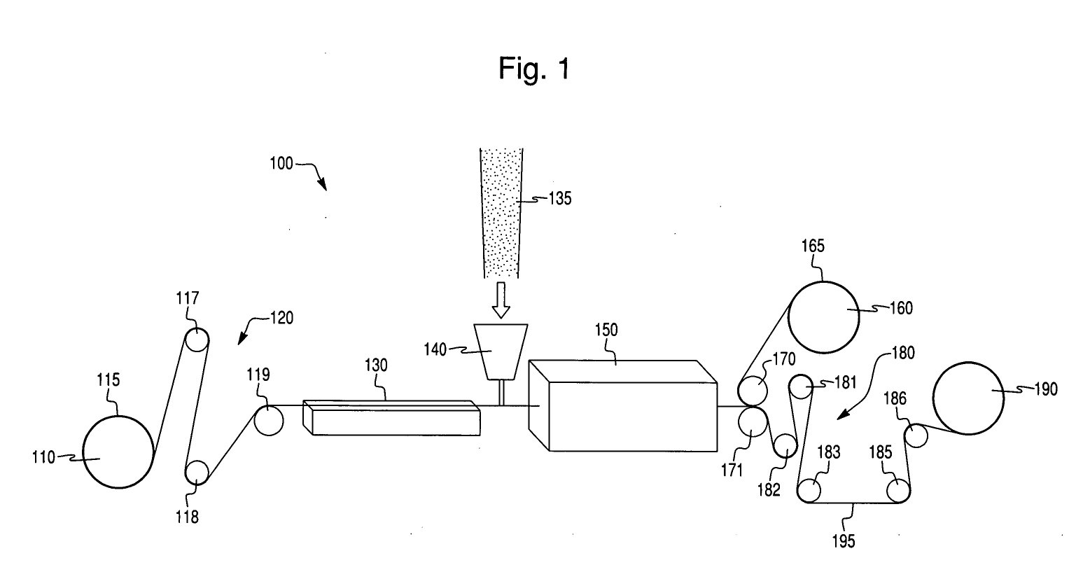

Looking now to FIG. 1, a process for making the deodorizing device is shown using a line device 100. The bottom nonwoven layer 115 is supplied on a roller 110 and fed into the line device 100. The bottom layer 115 passes through an accumulator 120 formed of three rollers, 117, 118, and 119 and is then fed through a spreader 130. The spreader 130 grips the bottom nonwoven layer 115 with needles and laterally pulls the bottom layer 115 off of the roller 110 and shapes the bottom layer 115 into a taut sheet.

Once the bottom layer 115 is taut it is ready to receive the deodorizing composition 135 containing the adhesive dispersed throughout. Hopper 140 contains the deodorizing composition and is suspended over the bottom layer 115. The deodorizing composition 135 is applied through hopper 140 onto a running length of the bottom layer 115 in proportional relation to the movement of the line device 100. Thus, as the bottom layer 115 moves the deodorizing composition 135 is deposited on the bottom layer 115. The line device 100 pulls the bottom layer 115 at a suitable speed to allow the proper loading of the deodorizing composition 135, e.g. 3-4 yards per minute. Too slow a line speed will result in higher loading of the deodorizing composition 135 onto the bottom layer 115.

Suitable spread rate ranges at which the deodorizing composition 135 are deposited onto the bottom layer 115 is about 10-60 ounces per minute, preferably 20-50 ounces per minute and more preferably 30-45 ounces per minute. A suitable rate at which the deodorizing composition 135 is spread onto the bottom layer 115 is about 40 ounces per minute. For instance, where the bottom layer 115 is 36 inches wide, and moving at a 4 yard per minute rate, an even coating of 10 ounces per square yard of the deodorizing composition 135 may be obtained with a coating level being equal to 0.22 grams per square inch. Coating weights of about 0.1-1.0 grams per square inch can be provided by controlling composition flow through hopper 140 and the line speed of bottom layer 115.

The following equation is used to calculate the predicted loading of the deodorizing composition 135, usually in the form of a powder, onto the bottom layer 115:

$$Load ( oz . \text{/} yd {.}^{2} ) = \frac{Spread rate ( oz . \text{/} min . )}{Speed ( yd . \text{/} min . ) \times Width ( yd . )}$$

After the powdered deodorizing composition 135 is applied, the bottom layer 115 coated with the deodorizing composition 135 passes through the curing oven 150. The oven 150 provides heat to obtain a temperature that is hot enough to melt the adhesive, but not too hot to cause damage to the bottom layer 115. A suitable temperature for the process is a temperature of about 100-300° F., and preferably about 270-300° F.

Immediately after leaving the oven 150, the top layer 165 is applied onto the bottom layer 115 containing the melted adhesive of the deodorizing composition 135. The top layer 165 is disposed on a roll 160 and suspended above the line device 100 just outside the oven 150. The two layers 115, 165 are pressed together through pressure rollers 170, 171, while layer 115 is still warm, under 50-200 pounds of pressure to seal layers 115 and 165 together and form a completed deodorizing device 195. After passing through pressure rollers 170, 171, the deodorizing device 195 pass through a second accumulator 180, formed by rollers 181, 182, 183 and then through tension rollers 185, 186 and is finally tensioned and rolled up on a roller 190. The completed deodorizing device 195 waits on the roller 190 ready to be manipulated (e.g. rolled, folded or reworked) into workable sizes for use in any end application.

Due to deformation incurred during the coating process, the completed deodorizing device 195 may have a slightly different thickness than the measurements specified for its individual parts.

Self-sealing mechanisms can be used when the deodorizing device is cut to prevent the deodorizing composition from being lost from the edges. Examples of specialized cutting mechanism used to seal edges of the deodorizing device include ultrasonic or thermal cutting devices. If such specialized cutting mechanisms are not used, then the adhesive component in the composition should optionally be increased to about 5-15 wt. %, preferably about 5-10 wt. % to insure sealing of the top and bottom sheets and adherence of the deodorizing composition to the sheets.

The deodorizing device may be used in a variety of deodorizing applications including vacuums (e.g. vacuum cleaner bags, vacuum cleaner dust caps), closets, gym bags, wardrobes, dresser drawers, dust masks, cars, planes, trains, pillows, bedding, mattresses, clothing, and other spaces where odors accumulate.

Examples disclosed below illustrate the present invention.

### EXAMPLE 1

Tests were conducted using Eureka® Model 402 vacuum cleaners. The Eureka® Model 402 vacuum cleaners were bagless and contained a cone shaped filter within the dust cup. A control filter was compared to a treated filter. The control filter was a standard untreated filter. The treated filter contained the deodorizing device of the present invention.

The deodorizing device included the deodorizing composition disposed between two nonwoven gas porous materials. The deodorizing composition contained about 91.5% sodium bicarbonate, 3.5% zeolite and 5% adhesive (Griltex®) and was coated on a 6.25″ x 6.25″ in size filter at a coating level of 8.7 oz./yd², (0.19 g/in²). The first nonwoven material was a moldable polyester having a fabric weight of 3.2 oz.yd², 108.5 g/m²; tensile strength MD of 25 lbs; tensile strength CD of about plus 75 lbs; thickness of 74 mils, 1.88 mm. The first nonwoven material served as the bottom layer of the deodorizing device and was a heavier sponge-like nonwoven material than the top layer. A suitable example of the first nonwoven material was obtained under the trade designation PN232 available from Precision Custom Coatings LLC, Totowa, N.J.

The second nonwoven material used herein was a hydrophilic PES/rayon having a fabric weight of 0.75 oz.yd², 25.4 g/m²; tensile strength MD of 10.8 lbs; tensile strength CD of 0.5 lbs; thickness of 8 mils, 0.203 mm. The second nonwoven material served as the top layer of the deodorizing device. The second nonwoven material was a “scrim” that minimized dustiness of the product and allowed for good air flow. A suitable example of the second nonwoven material was obtained under the trade designation PC757, available from Precision Custom Coatings LLC, Totowa, N.J.

Each of the filtered dust cups was filled with soil that was enhanced to emit a noticeable household odor. The soil consisted of 50 grams of damp vacuum cleaner dust, 2.5 grams of cat urine (provided by Martin Creek Kennels, Williford, Ark.) and 1.25 grams of Limburger cheese. The cat urine provided a strong, characteristic pet odor and the Limburger cheese imitated human body odor and strong kitchen odors.

The Eureka® Model 402 machines were equally loaded with soils and allowed to sit for 4 hours, and then placed inside cleaned new 30 gallon plastic garbage cans. The cans were used to contain the air emitted from the exhaust of the vacuum. The plastic lids on the garbage cans had sniffing ports cut into them so panelists could sample the air.

After the 4 hour gestation time, the lids were sealed and the vacuums were activated for 5 seconds. The air ejected in this 5 second period represents the most odorous air, usually encountered at machine startup. It was captured in the cans for panelists to sample.

20 panelists rated the two cans on a 0 to 6 scale for malodor. A rating of 0 represented least odor, a rating of 6 represented most odor. The following table shows the result from two separate trials:

TABLE 1

| Trial | Control | Treated |
|---|---|---|
| #1 | 3.6 | 1.4 |
| #2 | 3.8 | 1.1 |

In the test, the difference between the treated and control was statistically different at the 99% confidence level.

### EXAMPLE 2

Odor levels were measured in a sensory panel comparing a control sample to two treated samples. The odor studied in this test was 100 grams of damp vacuum cleaner dust. The tests were conducted by disposing the odors inside a 2 quart enclosed space.

The control sample had only the odor in the container. The two treated samples had the odors in the container and the deodorizing devices of the present invention where 7.4 grams of the deodorizing composition were loaded between two 6.25″ square pieces of nonwoven material. The first “treated” sample contained 88% sodium bicarbonate, 7% Smellrite®, and 5% Griltex® hotmelt on the nonwoven material. The second “treated” sample contained 91.5% sodium bicarbonate, 3.5% zeolite and 5% adhesive on the nonwoven material.

18 panelists smelled the odor inside the 2-quart enclosed space and rated the the smells on a 0 to 6 scale for malodor. A rating of 0 represented least odor, a rating of 6 represented most odor. The following table shows the result from the present test:

TABLE 2

| Sample | Odor level |
|---|---|
| Control | 4.2 |
| Treated sample #1 | 2.8 |
| Treated sample #2 | 2.4 |

There was no statistically significant difference between the two samples and both of the treated samples had a statistically significant lower odor source than the control.

What is claimed is:

1. A deodorizing device comprising:
a first gas porous sheet serving as a bottom layer of the deodorizing device;
a second gas porous sheet adhered to the first gas porous sheet, said second gas porous sheet serving as the top layer of the deodorizing device; and
a deodorizing composition disposed between the first gas porous sheet and the second gas porous sheet, said deodorizing composition containing an adhesive to secure said first to said second sheets.

2. The deodorizing device of claim 1, wherein the first and the second gas porous sheets are formed of non-woven thermoplastic fibers.

3. The deodorizing device of claim 2, wherein the thermoplastic fibers are selected from the group consisting of polyesters, polyolefins, nylon, rayon and blends thereof, including blends thereof which contain cotton.

4. The deodorizing device of claim 1, wherein the deodorizing composition contains a component selected from the group consisting of sodium bicarbonate, activated charcoal, activated carbons, clays, diatomaceous earths, cyclodextrin, quaternary ammonium salts, silane quaternary ammonium salts, fragrance oils and zeolites.

5. The deodorizing device of claim 1, wherein the device has a thickness range of about 0.50 mm-5 mm.

6. The deodorizing device of claim 1, wherein the deodorizing composition includes about 2-15 wt. % of said adhesive.

7. The deodorizing device of claim 1, wherein the first gas porous sheet is formed of polyester.

8. The deodorizing device of claim 1, wherein the second gas porous material is hydrophilic.

9. The deodorizing device of claim 1, wherein the deodorizing composition is provided relative to said first gas porous sheet at about 0.1 to 1.0 grams per square inch.

10. The deodorizing device of claim 1, wherein the first gas porous sheet has a thickness greater than said second gas porous sheet.

11. The deodorizing device of claim 1, wherein the deodorizing composition includes sodium bicarbonate, and optionally a zeolite.

12. The deodorizing device of claim 1, wherein said adhesive is a hotmelt adhesive.

13. A method of producing a deodorizing device comprising the steps of:
(a) supplying a first gas porous sheet material;
(b) applying a deodorizing composition on the first gas porous sheet material;
(c) adding adhesive particles to the deodorizing composition;
(d) activating the adhesive particles; and
(e) disposing a second gas porous sheet material over the first gas porous sheet material possessing the deodorizing material and the activated adhesive particles so as to adhere the first sheet to the second sheet.

14. The method of producing the device of claim 13, wherein the adhesive is a hotmelt type adhesive and said activating is done by heating.

15. The method of producing the device of claim 13, wherein the deodorizing composition and the adhesive particles are applied to a running length of said first gas porous sheet at a rate of about 10-60 ounces per minute.

16. The method of claim 14, wherein the adhesive particles are heated from 100-300° F.

17. The method of claim 13, wherein the deodorizing composition is applied as a mixture with said adhesive to said first porous sheet.

18. The method of claim 13, wherein the second gas porous sheet material is pressed to the first gas porous sheet material possessing the deodorizing composition and activated adhesive particles to form the deodorizing device.

19. The method of claim 13, wherein the deodorizing composition contains a component selected from the group consisting of sodium bicarbonate, activated charcoal, activated carbon, clays, diatomaceous earths, and zeolites.

20. The method of claim 13, wherein the deodorizing composition is applied on said first gas porous sheet at about 0.1 to 1.0 grams per square inch.
